(19) United States
(12) Patent Application Publication
(10) Pub. No.: US 2021/0291020 A1
(43) Pub. Date: Sep. 23, 2021

(54) REMOTE EXERCISE REHABILITATION MANAGEMENT SYSTEM

(71) Applicant: CHENGDU SHANGYI INFORMATION TECHNOLOGY CO., LTD., CHENGDU (CN)

(72) Inventors: Xi CHEN, CHENGDU (CN); Wen ZHOU, CHENGDU (CN); Runwen QI, CHENGDU (CN); Wenxia WANG, CHENGDU (CN); Li CHEN, CHENGDU (CN); Yanyan YUAN, CHENGDU (CN)

(73) Assignee: CHENGDU SHANGYI INFORMATION TECHNOLOGY CO., LTD., CHENGDU (CN)

(21) Appl. No.: 17/015,222

(22) Filed: Sep. 9, 2020

(30) Foreign Application Priority Data
Mar. 19, 2020 (CN) 202010196314.9

(51) Int. Cl. A63B 24/00; A63B 71/06; G16H 20/30; G16H 40/67; G16H 10/60

(52) U.S. Cl. CPC A63B 24/0075; A63B 71/0622; G16H 20/30; G16H 40/67; A63B 2022/0094; A63B 2024/0065; A63B 2220/62; A63B 2230/04; G16H 10/60

(57) ABSTRACT

The disclosure discloses a remote exercise rehabilitation management system for people with chronic diseases, including a server terminal, a doctor terminal and a patient terminal. Multi-disease integration principles are built in the server. Multiple independent fitness assessment protocol and multiple exercise rehabilitation prescriptions suitable for patients with different chronic diseases are stored in the server. The patient terminal collects the patient information by a health and lifestyle questionnaire, and transmits the patient information to the server. The server will design a fitness assessment protocol according to the patient's health information, and sends the fitness assessment protocol to the patient terminal. The patient terminal is able to present the fitness assessment protocol and collect patient's feedback. According to the feedback and built-in principles, the server will design an exercise prescription suitable to patient's health conditions.

### CROSS-REFERENCE TO RELATED APPLICATIONS

This application claims priority to Chinese Patent Application No. 202010196314.9 with a filing date of Mar. 19, 2020. The content of the aforementioned applications, including any intervening amendments thereto, are incorporated herein by reference.

### TECHNICAL FIELD

The disclosure relates to the technical field of exercise rehabilitation, and particularly relates to a remote exercise rehabilitation management system for people with chronic diseases.

### BACKGROUD OF THE PRESENT INVENTION

Exercise rehabilitation is one of the important ways to promote health conditions of people with chronic diseases. It involves an individualized exercise and lifestyle modification program, and provides a support system to help patients take control of risk factors, monitor progress, communicate with doctors, and adopt lifelong healthy behaviors.

Remote exercise rehabilitation is similar to traditional forms of rehabilitation but delivers it in a more convenient way. Like traditional rehabilitation, it includes a combination of exercise training, education and coaching to help patients recover and adopt healthier habits. The key messages are also the same, including eating healthy, staying active, managing stress, quitting smoking and taking medications as directed. Exercise rehabilitation program is also remotely monitored to help patients safely increase their activity levels.

The existing remote health manage systems in the market developed to support patient with single disease or condition, such as diabetes. However, most of diabetic patients not only suffer from diabetes but also other chronic diseases, such as obesity, hypertension and coronary heart disease. There was none software been found that can remotely delivery exercise rehabilitation to patients among with multiple chronic diseases. Thus, a remote exercise rehabilitation management system was developed to support people with chronic diseases to conduct a safe and effective exercise rehabilitation.

### SUMMARY OF PRESENT INVENTION

To compensate for the lack of a personalized, quantifiable remote exercise rehabilitation system to support patients among with multiple chronic diseases, the disclosure discloses a remote exercise rehabilitation management system for people with chronic diseases. The management system of the disclosure performs management according to diagnoses, health conditions and assessment results of the patient. Furthermore, the remote exercise rehabilitation management system is not only provide rehabilitation prescription to the medical professionals but also allows them to add, edit and modify the rehabilitation prescription, so as to realize convenient, flexible and intellectualized exercise rehabilitation management to the greatest extent. It is applicable to multiple scenes such as rehabilitation in hospital, rehabilitation in outpatient service and home-based rehabilitation.

The above objectives are achieved through the following technical solutions:

The multi-disease integration exercise rehabilitation management system includes a server terminal, a doctor terminal and a patient terminal;

In the server terminal, the principles of exercise rehabilitation management system were built. Fitness assessments and exercise prescriptions for individual and multiple diseases are designed and stored in the server terminal. According to the patient's health conditions, the system is able to create a fitness assessment protocol for the patient and design an exercise prescription based on the health conditions and assessment results.

The doctor terminal allows medical professionals to modify/edit the exercise prescription and send it to the patient after confirmation. The system also supports the medical professionals to prescribe exercise prescriptions according to a patient's medical information and results of assessment directly. The server will transmit the relevant data from the doctor terminal to the patient terminal after the exercise prescription is finally confirmed by the medical professionals.

The patient terminal is used for collecting patient information and assessment items and exercise prescriptions transmitted by the server and plays the received exercise videos of the exercise prescription.

Preferably, the principle of multiple chronic disease rehabilitation management system comprises applicable items for the health condition, safety principles and effective principles and remove inapplicable items for the health conditions.

Preferably, the rehabilitation program designed by the system includes a combination of exercise rehabilitation principles, exercise videos, follow-up plans, safety management and patient education.

Preferably, the fitness assessments are designed by combining multiple tagged movements in the exercise library. The tags include applicable tags and inappropriate tags.

Preferably, exercise prescription includes several independent exercise instruction videos and exercise pictures. Each of them has multiple tags from different dimensions. The tags include applicable tags and inappropriate tags.

Preferably, the exercise prescription principle is the basic principle of exercise prescription for special population based on evidence-based medicine, including exercise frequency, exercise intensity, exercise time, exercise type, total amount of exercise, exercise progression and exercise mode.

Preferably, the follow-up plans include questionnaires, scales, medication records, measurements, and examination reports.

Preferably, the patient terminal is able to connect with medical devices, such as heart rate monitor, electronic digital blood pressure monitor, electrocardiogram devices, to monitor patients' vital signs.

Preferably, patient education includes health information related to diagnoses and measurements; the patient education is presented in text, audio, pictures and video.

Preferably, the exercise safety reminder is developed to maintain patient safety throughout the whole process of exercise, including before, during, and after exercise.

Compared with the existing technology, the disclosure has the following beneficial effects:

1. The multi-disease integration exercise rehabilitation management system includes a server terminal, a doctor terminal and a patient terminal. The server has built-in multi-disease rehabilitation principles, while the server stores several independent fitness assessments and exercise rehabilitation protocols for patient with/without multiple chronic diseases. Based on the patient basic and medical information collected from the patient terminal, the server is able to select and send appropriate fitness assessments to the patients. The doctor terminal reviews and modifies the sports rehabilitation guidance program and finally confirms that it is sent to the appropriate patient terminal. Compared with the prior technology, the server of the present invention can integrate a number of exercise rehabilitation programs for a single disease according to the actual condition of the patient, so that the patient's motor rehabilitation program conforms to the actual disease and ensures the safety and effectiveness of the patient during the exercise. The doctor terminal allows medical professionals to modify/edit the exercise prescription and send it to the patient after confirmation. Compared with the existing technology, the multi-disease integration exercise rehabilitation management system ensures the safety and effectiveness of the patient in the process of rehabilitation.

2. The principle of multiple chronic disease rehabilitation management system comprises applicable items for the patient's health condition, safety principles and effective principles and remove inapplicable items for the patient's health conditions. Applicable items mean all of the exercise items suitable for the patient's diagnoses and health conditions according to the published literatures. Inapplicable items mean all of the exercise items inapplicable and risky for the patient's diagnoses and health conditions according to the published literatures. Safety principle is to ensure the safety first when the exercise prescription and the safety principle are in conflict. The effectiveness principle is to ensure that the exercise prescription is effective for patients when there is no conflict in safety-The safety, effectiveness and performability of the integrated exercise rehabilitation guidance scheme are ensured through comprehensive selection of the above principles.

3. Exercise prescription includes several independent exercise instruction videos and exercise pictures. Each of them has multiple tags from different dimensions. The tags include applicable tags and inappropriate tags. The system uses applicable tags to filter out fitness assessment that are suitable for patient basic and clinical information, and uses inappropriate tags to eliminate inapplicable fitness assessment. This can not only ensure the effectiveness and safety of the fitness assessment, but also avoid the risk brought to patients by inappropriate fitness assessment.

4. The rehabilitation program designed by the system includes a combination of exercise rehabilitation principles, exercise videos, follow-up plans, safety management and patient education, so as to comprehensively cover the relevant contents involved in the exercise rehabilitation. Compared with other products, the function are very comprehensive. The designated exercise rehabilitation program are fit to the actual health condition of the patient, so as to improve the curative effectiveness and safety of the exercise rehabilitation program.

5. The integration of the exercise prescription principle includes exercise frequency, exercise intensity, exercise time, exercise type, total exercise amount, exercise progress and exercise mode. It is applicable to different health conditions. The exercise prescription principle for individual disease or health condition is based on evidence-based medicine evidences. The patient with multiple diseases and health conditions are combined according to the above applicable items combination principle, the inapplicable items removal principle, the safety principle and the effectiveness principle. It is ensured that the exercise prescription principle of the patient is safe and effective under the complicated condition; compared with the other existing technologies.

Meanwhile, the disclosure also comprises integration of exercise rehabilitation videos. Each of the videos has multiple tags from different dimensions. The tags include applicable tags and inappropriate tags. The system uses applicable tags to filter out exercise videos that are suitable for patient basic and clinical information, and uses inappropriate tags to eliminate inapplicable exercise videos. This can not only ensure the effectiveness and safety of exercise prescription, but also avoid the risk brought to patients by inappropriate exercise movements. Compared with other existing rehabilitation program, the rehabilitation program of the system is more targeted and more conforms to the actual rehabilitation needs of the patient.

6. The follow-up plans include questionnaires, scales, medication records, measurements, and examination reports. This meets the various needs of clinical daily follow-up and scientific research follow-up. Moreover, it is convenient for medical staff to adjust the treatment plan at any time according to the actual situation of the patient, which is beneficial to improve the safety and effectiveness of the patient's exercise rehabilitation. This system combines the follow-up plans for various diseases and health conditions according to the above applicable items combination principle, the inapplicable items removal principle, which can meet different follow-up needs.

7. The patient terminal is able to connect with medical devices, such as heart rate monitor, electronic digital blood pressure monitor, electrocardiogram devices, to monitor patients' vital signs. The above-mentioned devices collect the physiological index data of the patient in real time. This can help patients understand their exercise intensity and understand whether their physiological indicators are within the safe limits. The data can also be used as the basis for doctors to adjust exercise prescription to further improve the exercise prescription.

8. The exercise rehabilitation program of the disclosure includes exercise safety reminder and patient education. The patient education includes health knowledge adapted to the patient's diagnoses and fitness assessment results. The patient education is presented in text, audios, pictures and videos. When multiple diseases and health conditions are combined, the system combines the patient education information for various diseases and health conditions according to the above applicable items combination principle and the inapplicable items removal principle, which can meet different patients education needs.

9. The disclosure includes the integration of exercise safety reminders, which include the safety reminder before exercise, the safety reminder during the exercise and the safety reminder after exercise. The exercise safety reminders are able to interact with the patients. The exercise video will not be displayed when biometric data input by the patient is not in the safe range before exercise. The exercise video will be paused when the real-time biometric data exceeds the safe range during exercising. If the patient input score of Borg rating of perceived exertion or the biometric data exceeds the safety limits after exercise, the server will remind the doctor to contact the patient and modify the prescription through the doctor terminal. It greatly guarantees the safety of the patients. When multiple diseases and/or health conditions are combined, the interactive safety reminders are also combined according to the above applicable item combination principle and inapplicable item removal principle, so as to ensure that the safety of the patients. Compared with the traditional exercise rehabilitation program, the repetitive work of the doctor is reduced, and the patient does not need to remember a lot of safety precautions.

### DESCRIPTION OF THE DRAWINGS

FIG. 1 is a topological graph of the disclosure.

FIG. 2 is a flowchart of the disclosure.

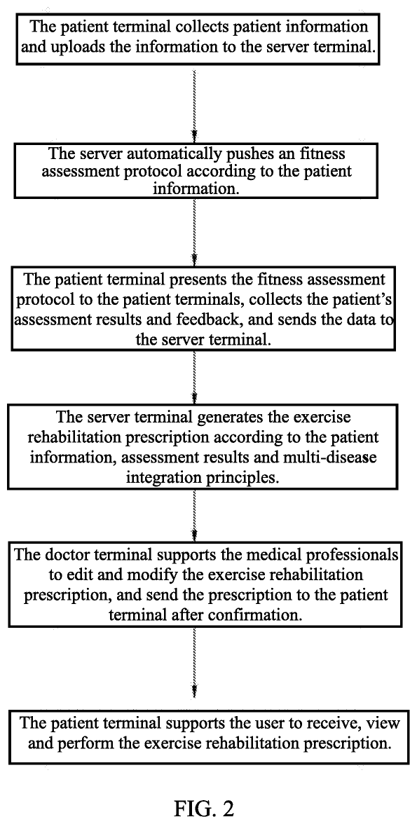

FIG. 3 is a topological graph according to embodiment 2 of the disclosure.

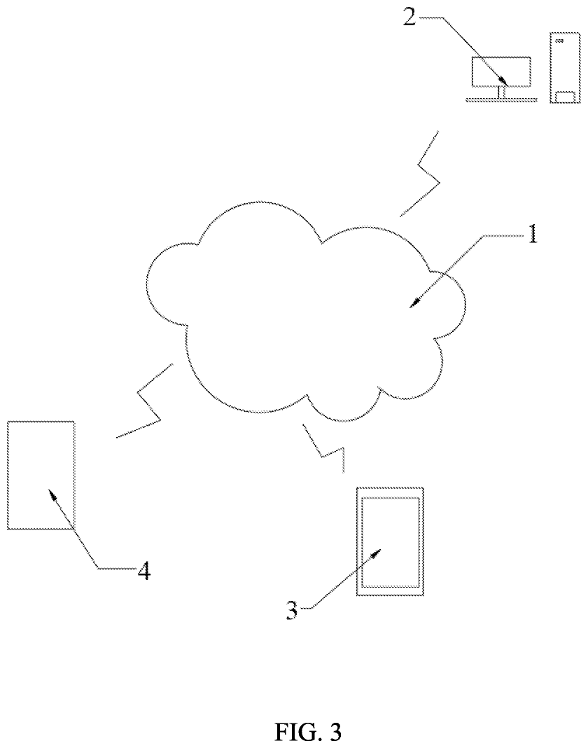

FIG. 4 is a topological graph according to the disclosure.

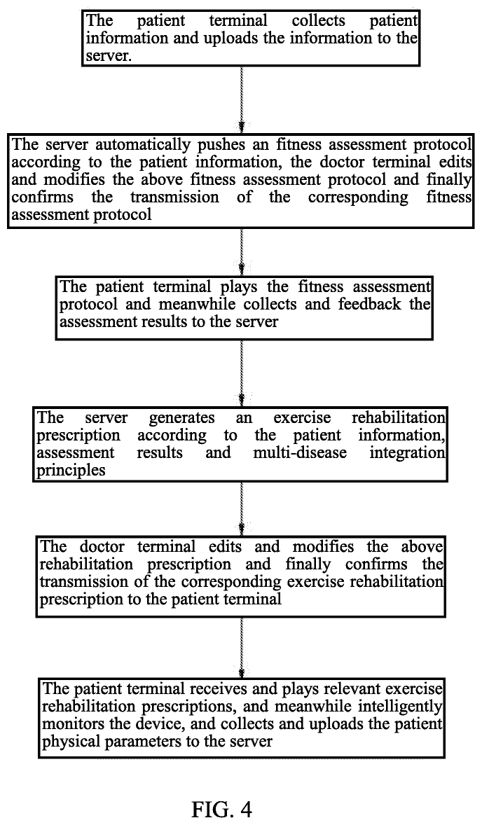

Reference numbers: 1, server; 2, doctor terminal 3, patient terminal; 4, intelligent monitoring devices.

### DETAILED DESCRIPTION OF PREFERRED EMBODIMENTS

The present utility model will be further described in combination with figures and embodiments. The implementations of the present utility model include but are not limited to the following embodiments.

### Embodiment 1

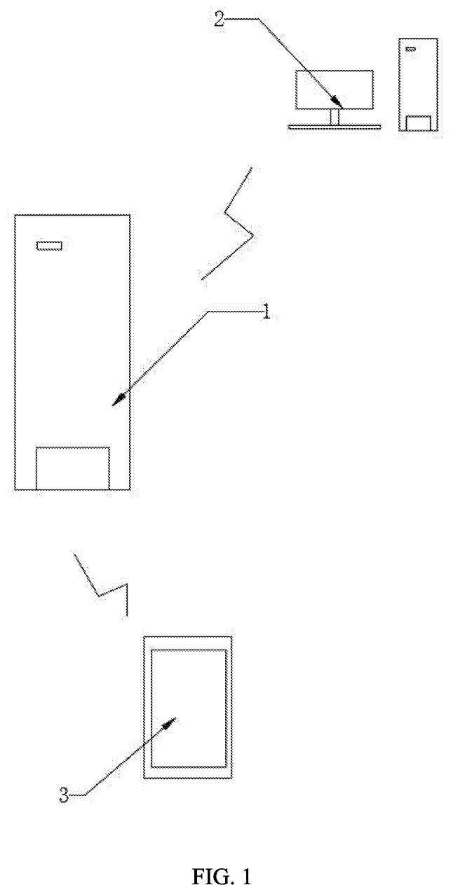

The embodiment, a basic embodiment of the disclosure, discloses a multiple chronic disease rehabilitation management system, as shown in FIG. 1. It includes a server 1, a doctor terminal 2 and a patient terminal 3. The server is built on the cloud server. The doctor terminal 2 and the patient terminal 3 are built on the PC terminals, smart phones or other intelligent devices which have relevant control software programs. The server 1 is communicated with the doctor terminal 2 and the patient terminal 3 through the Internet. The patient terminal 3 is bound with the unique doctor terminal 2. The doctor terminal 2 can be simultaneously bound with multiple patient terminals 3. The patient terminal 3 are communicated with the doctor terminal 2 through the Internet.

The multi-disease integration principles, including the applicable items combination principle, the inapplicable items removal principle, the safety principle and the effectiveness principle, are built in the server 1. The applicable items combination principle means that the system will screen out all rehabilitation programs suitable for the patient with multiple chronic disease or health conditions, and will remove same items and combine different items in different programs. The content of the final rehabilitation program includes exercise prescriptions, follow-up plans, exercise safety reminders and patient education. For example, patient with knee arthritis will receive an exercise rehabilitation program A for knee arthritis, and patient with diabetes will receive an exercise rehabilitation program B for diabetes, patient with knee arthritis and diabetes will receive an exercise program combining exercise program A and program B;

For another example, the follow-up plan for patients with diabetes includes: measure fasting blood glucose twice a week and have vascular artery color Doppler ultrasound once a year. The follow-up plan for patients with hypertension includes: measure blood pressure three times a week and have vascular artery color Doppler ultrasound once a year; the follow-up plan for patients with diabetes and hypertension will include: measure fasting blood glucose twice a week, measure blood pressure three times a week and have vascular artery color Doppler ultrasound once a year.

The inapplicable item removal principle is remove items that are risky or not suitable for patient with specific diseases or health conditions. For example, patients with hypertension are recommended do not have exercise in a head-down position. Obese patients have no such concerns, but not recommended to have jumping movements. For patient with both diabetes and obesity, the exercise prescription will not contain jumping movements and movements performed in a head-down position.

The safety principle is to ensure the safety first when the exercise prescription and the safety principle are in conflict. For example, the recommended exercise intensity for patients with diabetes is moderate to high intensity, the total exercise time amount is 75-150 minutes per week. The recommended exercise intensity for patient with hypertension is moderate, the total exercise time amount is 150 minutes per week. For patient with both diabetes and hypertension, the system recommended exercise prescription is in moderate intensity, and total exercise amount is 75-150 minutes per week.

The effectiveness principle is to ensure that the exercise prescription is effective for patients when there is no conflict with safety principles. For example, the recommended exercise intensity for patients with hypertension is moderate, the total exercise time amount is 150 minutes per week, and the recommended exercise intensity for obese patients is moderate, the total exercise time amount is 250 minutes per week. The exercise prescriptions for patients with both hypertension and obesity have no conflict in safety principles, so the recommended exercise prescription for the patients with both diseases is moderate intensity exercise, and total exercise time amount is 250 minutes per week.

In the server, exercise prescription includes several independent exercise instruction videos and exercise pictures. Each of them has multiple tags from different dimensions. The tags include applicable tags and inappropriate tags. The system uses applicable tags to filter out fitness assessment that are suitable for patient basic and clinical information, and uses inappropriate tags to eliminate inapplicable fitness assessment. The rehabilitation program designed by the system includes a combination of exercise rehabilitation principles, exercise videos, follow-up plans, safety management and patient education. The applicable tags include age, gender, exercise habits, diagnoses and pain area. The inapplicable tags include disease diagnoses, pain area, operation method and operation time. The server will design the fitness assessment protocol according to patient's information, including age, gender, exercise habits, and diagnoses) by using applicable tags and inapplicable tags. The server will send the fitness assessment to the patient terminal and collect patient's assessment results. According to assessment results, the system will determine the patient's health and fitness status. Based on this, the server will design an exercise rehabilitation program, including exercise prescription principles, exercise videos, follow-up plans, safety reminder and patient education, and send it to the patient terminal.

The exercise prescription principle includes exercise frequency, exercise intensity, exercise time, exercise type, total exercise amount, exercise progress and exercise mode. When the patient has only a single disease or health condition, the system will create rehabilitation program based on evidence-based medicine evidence, safety principles and effective principles. While, when the patient has multiple chronic diseases or health conditions, the system will perform the integration principles according to diagnoses and health conditions of patients, the safety principle and the effectiveness.

Exercise rehabilitation instructions include several independent exercise instruction videos and pictures. Each item has a name, a voice instruction, a video or a picture. Each motion video or motion picture carries multiple tags from different dimensions. Filter tags include applicable tags and not applicable tags. Applicable tags include type, strength, and location. Inapplicable tags include pain site, diagnosis of disease, method of operation, and data of operation. The patient with multiple diseases and health conditions are combined according to the above applicable items combination principle, the inapplicable items removal principle, the safety principle and the effectiveness principle.

The follow-up plans include questionnaires, scales, medication records, measurements, and examination reports. This meets the various needs of clinical daily follow-up and scientific research follow-up. Moreover, it is convenient for medical staff to adjust the treatment plan at any time according to the actual situation of the patient, which is beneficial to improve the safety and effectiveness of the patient's exercise rehabilitation. This system combines the follow-up plans for various diseases and health conditions according to the above applicable items combination principle and the inapplicable items removal principle.

The patient education includes health knowledge related to the patient's diagnoses and fitness assessment results. The patient education is presented in text, audios, pictures and videos. When multiple diseases and health conditions are combined, the system combines the patient education information for various diseases and health conditions according to the above applicable items combination principle and the inapplicable items removal principle, which can meet different patient education needs.

The terminal has developed features of safety reminders, including the safety reminder before exercise, the safety reminder during the exercise and the safety reminder after exercise. The exercise safety reminders are able to interact with the patients. Before exercise, the patient will be suggested to input their biometrics. The exercise video will not be displayed when biometrics (definitely heart rate, dependently blood pressure and blood glucose) are not in the safe range before exercise, and remind the patient to take a rest or visit the doctor. During exercise, the exercise video will be paused when the real-time data of biometrics in exercise exceeds the safe range and remind the patient. For example, a patient's heart rate exceeds the safe range, the terminal will display a reminding voice: “your heart rate is very high, please slow down your pace” and pause the exercise video. After finishing each exercise session, the terminal will ask the patient's feedback of exercise, including score of Borg rating of perceived exertion, biometrics and symptoms. If the input data exceeds the safety limits or indicates that the patient is at risk, the terminal will remind the patient's doctor to contact the patient and modify the prescription through the doctor terminal, so as to ensure that the safety of the patients. The traditional exercise rehabilitation guidance requires doctors to give the patient an orientation and supervise the patient's exercise according to the patient's condition. Compare with the traditional rehabilitation, this disclosure reduces the repeated work of doctors and reduces the burden of remembering the notes of different diseases. Compared with the exercise safety prompt for the single disease, monitoring and reminding patient's condition by monitoring different biometrics is more suitable for actual clinical demands;

The medical professionals are supported to check, modify and edit the exercise rehabilitation prescription automatically generated by the server in the doctor terminal. On one hand, the errors and omissions in the exercise rehabilitation prescription can be corrected manually; on the other hand, the revision of medical staff can make the exercise rehabilitation prescription more suitable for the actual situation of patients and improve the curative effect. After the medical professional's confirmation, the server will transmit corresponding exercise rehabilitation prescription to the patient terminal.

The patient terminal is used for collecting and uploading the patient information to the server and meanwhile taking charge of receiving and demonstrating the fitness assessment protocol, and assisting the patient to feedback the corresponding fitness assessment after completing it. The patient terminal also receives the exercise rehabilitation prescription transmitted by the server, and display the corresponding contents to the patient.

### Embodiment 2

This embodiment, as a preferred embodiment of the disclosure, discloses a remote exercise rehabilitation management system for people with chronic diseases on the basis of embodiment 1, as shown in FIG. 1, including a server 1, a doctor terminal 2 and a patient terminal 3. The server 1 adopts a cloud server, and the doctor terminal 2 and the patient terminal 3 are PC terminals, smart phones or other intelligent devices which have relevant control software programs, wherein the server 1 is communicated with the doctor terminal 2 and the patient terminal 3 respectively through the Internet. The patient terminal 3 is bound with the unique doctor terminal 2. The doctor terminal 2 can be simultaneously bound with multiple patient terminals 3. The communication between the patient terminal 3 and the doctor terminal 2 is realized through the Internet. The patient terminal 3 also includes intelligent monitoring devices 4 for monitoring the biometrics of the patient. The intelligent monitoring devices 4 include a heart rate monitor, an electronic digital blood pressure monitor, electrocardiogram devices. The above intelligent monitoring devices 4 are able to communicate data with the server 1 in real time, and the server 1 receives and stores the data, which supports doctor to retrieve the data at any time, provide data basis for optimization of the exercise rehabilitation prescription and improve the effectiveness;

The doctor terminal supports the medical professionals to view, modify the fitness assessment and exercise prescription designed by the system. At the same time, the medical professional supported to design a new fitness assessment protocol, which can be marked with applicable and inapplicable tags. In addition, the doctor terminal supports the medical professionals to add movement videos or pictures, and mark them with applicable and inapplicable tags.

The patient terminal supports to connect with intelligent devices, such as heart rate monitor, electronic digital blood pressure monitor and electrocardiogram devices, for real-time vital signs detection for patients. When the real-time biometrics during the exercise exceeds the safety limit specified in the exercise prescription principle, the exercise video will be paused and the terminal will display a reminding voice: “your heart rate is very high, please slow down your pace” and pause the exercise video, in order to improve the safety of exercise rehabilitation of the patient.

When the multi-disease integration exercise rehabilitation management system is used, the patient terminal collects the patient information by a health and lifestyle questionnaire, and transmits the patient information to the server. The server will design a fitness assessment protocol according to the patient's health information, and sends the fitness assessment protocol to the patient terminal. The patient terminal is able to present the fitness assessment protocol and collect patient's feedback. According to the feedback and built-in principle. The server will design an exercise prescription suitable to patient's health conditions. The doctor terminal supports medical professionals to view and modify the exercise rehabilitation prescription customized by the system, and finally confirm to transmit the prescription to the patient terminal. The patient terminal will present the exercise prescription to the patient and collect feedback and biometrics. The medical professional is supported to review the patient's performance and data, to evaluate the value of treatments and optimize it.

Compared with the existing technology, the disclosure combines and integrates the exercise rehabilitation prescription applicable to different patient with chronic diseases, which meets the requirements of exercise rehabilitation for patients with multiple chronic diseases. It not only improves the curative effect, but also greatly improves the safety of the exercise rehabilitation program.

We claim:

1. A remote exercise rehabilitation management system for people with chronic diseases, comprises a server terminal (1), a doctor terminal (2) and a patient terminal (3); the multi-disease integration principles are built in the server terminal (1), multiple independent fitness assessments and multiple exercise rehabilitation prescription applicable to different single chronic diseases are stored in the server terminal (1); the server terminal (1) pushes corresponding fitness assessment protocol according to patient health information collected from the patient terminal (3) and record the assessment results; the patient terminal will design an exercise rehabilitation prescription according to the assessment results, patient health information and built-in multi-disease integration principles;
the doctor terminal (2) supports medical professionals to view and modify the exercise rehabilitation prescription customized by the server (1); after the above exercise rehabilitation prescription are finally confirmed by the medical professional, the doctor terminal (2) controls the server (1) to transmit relevant data to the patient terminal (3);
the patient terminal (3) is used for collecting patient information and fitness assessment results, and uploading these data to the server terminal(1); the patient terminal (3) receives fitness assessment protocol and exercise rehabilitation prescription transmitted by the server terminal (1) and plays the received exercise test schemes and exercise rehabilitation guidance schemes to the patient.

2. The remote exercise rehabilitation management system for people with chronic diseases according to claim 1, wherein the multiple chronic disease integration principles, including applicable items combination principle, the inapplicable items removal principle, the safety principle and the effectiveness principle;
the applicable items combination principle is that completely identical items in multiple exercise rehabilitation guidance schemes simultaneously applicable to different diseases and special situations of the patient are combined when the patient suffers from multiple diseases and special situations, and different items occur in parallel; namely, all the items in all the exercise rehabilitation guidance schemes are remained, the completely identical items only occur once, and different items occur in parallel;
the inapplicable item removal principle is that if there are any contents inapplicable to a disease in the exercise rehabilitation guidance schemes which have been screened, corresponding items are removed; namely, any items conforming to disease contraindications are all removed, for example a hypertensive patient should not do a head-down feet-high action, an obese patient is allowed to do the head-down feet-high action, and a hypertensive and obese patient is allowed to remove the head-down feet-high action;
the safety principle is that for different health conditions, the exercise rehabilitation guidance scheme has different rules for the same item, a certain disease has explicit safety stipulation on this item, and the safety is preferably ensured;
the effectiveness principle is that for different health conditions, the exercise rehabilitation guidance scheme has different rules for the same item, and all the diseases and health conditions of the patient have no safety stipulation for the item, so as to ensure that the item is effective to all the diseases.

3. The remote exercise rehabilitation management system for people with chronic diseases according to claim 1, wherein the exercise rehabilitation program, including the exercise prescription principles, exercise rehabilitation movement videos, a follow-up plan, exercise safety reminders and patient education.

4. The remote exercise rehabilitation management system for people with chronic diseases according to claim 1, wherein the fitness assessment protocol, which is designed with multiple tags from different dimensions; the tags include applicable tags and inappropriate tags.

5. The remote exercise rehabilitation management system for people with chronic diseases according to claim 1, wherein several independent exercise instruction videos and exercise pictures; each of them has multiple tags from different dimensions; the tags include applicable tags and inappropriate tags.

6. The remote exercise rehabilitation management system for people with chronic diseases according to claim 1, wherein the exercise prescription principle, which is based on the evidence-based exercise prescription principles, including exercise frequency, exercise intensity, exercise time, exercise type, total exercise amount, exercise progression and exercise mode.

7. The remote exercise rehabilitation management system for people with chronic diseases according to claim 1, wherein the follow-up plans, which include questionnaires, scales, medication records, measurements, and examination reports.

8. The remote exercise rehabilitation management system for people with chronic diseases according to claim 1, wherein the patient terminal (3), which is able to connect and communicate with intelligent monitoring devices (4); intelligent monitoring devices include heart rate monitor, electronic digital blood pressure monitor and electrocardiogram devices.

9. The remote exercise rehabilitation management system for people with chronic diseases according to claim 1, wherein the patient education, which is customized by the system according to the patient's diagnoses, assessment results and health conditions; the patient education is presented in forms of words, audios, pictures and videos.

10. The remote exercise rehabilitation management system for people with chronic diseases according to claim 1, wherein the exercise safety reminders, which includes safety reminders before exercise, safety reminders during the exercise and safety reminders after exercise.
